(19) United States
(12) Patent Application Publication
(10) Pub. No.: US 2004/0037859 A1
(43) Pub. Date: Feb. 26, 2004

(54) Formulation containing wax-esters

(76) Inventors: Georges Cecchi, Marseille (FR); Jacques Margnat, Peyruis (FR)

Correspondence Address: DENNISON, SCHULTZ & DOUGHERTY, 1745 JEFFERSON DAVIS HIGHWAY, ARLINGTON, VA, 22202, US

(21) Appl. No.: 10/398,281

(22) PCT Filed: Oct. 4, 2001
(86) PCT No.: PCT/FR01/03055

(30) Foreign Application Priority Data
Oct. 13, 2000 (FR) 00/13124

(51) Int. Cl. A61K 7/06

(52) U.S. Cl. 424/401; 424/74

(57) ABSTRACT

The present invention concerns a formulation comprising non-greasy emollients based on wax-esters the constituents of which have a molecular weight of less than 600 Daltons, preferably less than 450 Daltons, comprising:

esters of fatty acids and a fatty alcohol deriving from interesterification by a said fatty alcohol of triglycerides of an oil of natural origin, preferably vegetable origin; and

residual triglycerides, diglycerides and monoglycerides derived from said interesterification;

characterized in that it comprises at least two said wax-esters wherein at least one said wax ester is a hydrogenated wax ester comprising:

hydrogenated fatty acid esters derived from inter-esterification by a said fatty alcohol of triglycerides of an oil of natural origin, preferably vegetable origin, followed by hydrogenation of said esters; and

residual hydrogenated triglycerides, diglycerides and monoglycerides from said interesterification, followed by said hydrogenation.

The present invention relates to formulations containing non-greasy or non-fatty emollients based on wax-esters, in particular hydrogenated wax-esters.

More particularly, the invention relates to applications of such formulations in the cosmetics and pharmaceutical fields.

More precisely, the wax-esters of the present invention are wax-esters the constituents of which have a molecular weight of less than 600 Daltons, preferably less than 450 Daltons, comprising a mixture of:

a plurality of esters of fatty acids and a fatty alcohol deriving from interesterification by a said fatty alcohol of triglycerides of an oil of natural origin, preferably vegetable origin, optionally followed by hydrogenation of said esters;

residual triglycerides, diglycerides and monoglycerides, optionally hydrogenated, originating from said interesterification.

Emollients are widely used in cosmetics and in pharmaceuticals to render dry skin supple and to improve its elasticity. The term “emollient” generally means an ensemble of perceptions transmitted by touch and vision. Perceptions involving touch are softness, elasticity, and slide. Perceptions involving vision are shine and mattness.

Suppliers of cosmetic starting materials have proposed a considerable number of emollients. Such emollients are distinguished from one another by their chemical nature and also by the resultant of two factors: softness on application and residual softness. Emollients exist that have a protective effect, others have an overgreasing effect, while some give the impression of dryness and others act as astringents.

The vast majority of such emollients are characterized by the presence of fatty acids with a linear or branched carbon-containing chain of any length. Such fatty acids are themselves combined in the form of esters with alcohols containing a linear or branched carbon-containing chain of any length. Those esters and their fatty acids form the basis of the emollient effect. In general, it is considered that this category of emollient contains two families of esters: those with a completely natural origin, and those with a synthetic origin, synthesis involving esterification of the fatty acid by an alcohol. Synthesised esters are generally manufactured from saturated fatty acids, which endows them with greater stability to oxidation, but removes any possibility of integration into the biosynthetic process occurring in the epidermis. It is well known that polyunsaturated fatty acids (linoleic and linolenic) known as essential fatty acids can be transformed by the enzymes contained in the epidermis into other polyunsaturated fatty acids that can act, inter alia, to limit transepidermal water loss. That water loss limitation guarantees skin softness and that particular emollient effect is desired in esters of natural origin such as those found in vegetable oils and fats, marine oils, and certain animal fats.

All fats are constituted by a mixture of esters, which are triglycerides or triesters of glycerol and fatty acids. The nature of the fatty acids involved in those esters determines the consistency of fats resulting from mixing them. The richer the fats in saturated fatty acids, the harder their consistency, until fats or butters are obtained that are solid at 20° C. Products that are completely solid at that temperature can be obtained with completely hydrogenated fats. Conversely, the higher the (mono- and poly-) unsaturated fatty acid content, the greater the tendency of the fat to be completely fluid at 20° C.

The above is true for vegetable oils characterized by a fatty acid composition, the overall unsaturated fatty acid content of which is usually more than 85%. The liquid consistency of oils is a first advantage in obtaining an emollient effect. The effect of a liquid consistency is supplemented by that of essential fatty acids, such as the linoleic acid that is always present in vegetable oils in varying amounts which are a function of the botanical origin of the oleaginous species from which they derive. As mentioned above, transformation of that linoleic acid into other unsaturated fatty acids via a biosynthetic process results in a substantial moisturising effect contributing to keeping the epidermis soft. Finally, for vegetable oils, the substantial biological effect of their nonsaponifiable components such as squalene, carotenes, triterpenic alcohols, tocopherols and mainly phytosterols, must be taken into account. In contrast, such oils can be completely hydrogenated to produce solid emollient fats that have lost their biological activity, but which are highly stable to oxidation and can produce the consistency required for some creams.

While all of those advantages are well known, vegetable oils and fats nevertheless generally suffer from the major disadvantage of producing a greasy feel after application to the skin because of their low rate of penetration into the epidermis. In general, it is recognized that the rate of percutaneous penetration of a molecule is inversely proportional to its molecular weight. That rate can be considered to be relatively high for a molecular weight of 400 Daltons but beyond that point, it starts to diminish rapidly. Now, the molecular weight of vegetable oil triglycerides is centred around 870 Daltons, well beyond the 400 Dalton limit. Thus, it can be seen that vegetable oils such as those used in cosmetic and pharmaceutical compositions can leave a greasy impression supplied by the triglycerides which penetrate only very slowly into the skin.

Jojoba oil is known to be a natural constituent of wax-esters. However, the molecular weight of the two principal constituents is 612 Daltons, which means that the percutaneous absorption rate is slow. Further, the particular feature of that wax, composed of esters of mono-enic fatty acids and mono-enic fatty alcohols, means that by definition, it contains no essential fatty acids. Topical application, then, cannot benefit from the effect of essential fatty acids in limiting transepidermal water loss, as is the case for Ceresters® in general. Finally and especially, jojoba oil containing only wax-esters to the exclusion of monoglycerides does not have emulsifying properties.

The Applicant's unpublished French patent application FR 99/05006 and International patent application PCT/FR00/01901 describe a process for producing emollients wherein the molecular weight of the principal components, namely esters of fatty acids and fatty alcohols, is less than about 600 Daltons, preferably less than about 450 Daltons, which can produce an emollient preparation that does not feel greasy from vegetable oils and fats in general. That process consists of transforming vegetable oils or fats in general and purifying the transformation product under conditions so that the integrity of their fatty acids and their nonsaponifiable products are satisfied, to exploit all the properties of the fats without the disadvantage of a greasy feel.

That process comprises the following steps:

a) interesterification of triglycerides of a fatty substance, preferably of vegetable origin, using a primary alcohol, preferably of vegetable origin, in the presence of a catalyst;

b) elimination of the catalyst;

c) distillation of the residual alcohol, preferably in the presence of a decolorizing agent, then eliminating the decolorizing agent;

d1) either wintering the residue, preferably decolorized, to at least partially crystallize the residual glycerides; then eliminating said crystallized residual glycerides, in particular by filtering;

d2) or hydrogenating the preferably decolorized residue.

That process can transform triglycerides of fats, in particular those of vegetable oils, into much lower molecular weight molecules, which penetrate the epidermis more easily.

Step a) consists of alcoholysis (interesterification) of triglycerides with a fatty alcohol, the reaction giving rise to the formation of wax-esters chemically defined as esters of fatty acids and a fatty alcohol.

The wintering operation is carried out in step d1) by stirring the decolorized distillate at a temperature in the range about 10° C. to about 14° C., for a period that is generally at least about 1 hour to at most about 4 hours, after which the wintering product is filtered. The wintering temperature can be reduced but that would run the risk that a portion of the wax-esters of the invention would crystallize and then be eliminated with the crystallized residual glycerides.

In step d1), those residual glycerides that are crystallized are saturated mono-, di- or triglycerides resulting from incomplete interesterification by said primary alcohol in step a). Their elimination can produce products that are completely liquid at the wintering temperature, in particular generally liquid at ambient temperature, i.e., at a temperature of at least 15° C. After step d1), there remain unsaturated residual mono-, di- and triglycerides from said interesterification.

The products obtained in step d1) are hereinafter termed “non-hydrogenated wax-esters” or “Ceresters®”.

In step d2), hydrogenation of the residue results in products with higher melting points, generally solid at ambient temperature, and in general with a melting point in the range 23° C. to 80° C. depending on the molecular weight of the products. Those products are hereinafter termed “hydrogenated wax-esters” or “Phytowaxes®”.

In step d2), the product (residue) recovered after distillation of the residual alcohol can be hydrogenated in a reactor at a hydrogen pressure of about 1 bar to about 20 bars, in the presence of a catalyst such as a nickel-based or palladium-based catalyst, at a temperature of at least about 100° C. to at most about 220° C., for a period of generally at least about 2 hours to at most about 8 hours. Under these conditions, all the unsaturated bonds of the carbon-containing chain of the acid and the alcohol (if unsaturated) are hydrogenated, producing a hydrogenated product with an iodine number of less than 1. The catalyst is then separated by simply filtering through paper.

The alcohol used in the interesterification step can in particular be selected from C₁-C₂₂ alkanols, C₃-C₂₂ alkenols or branched C₃-C₂₂ alcohols. The branched alcohols are alcohols that can carry C₁-C₈ alkyl substituents. Preferred C₁-C₂₂ alkanols are C₄-C₁₈ alkanols, in particular C₆-C₁₈; preferred branched C₃-C₂₂ alcohols are C₈-C₂₂ alcohols.

Advantageously, in step a), about 30% by weight to about 150% by weight of alcohol is used with respect to the weight of fatty substance. At the end of the inter-esterification reaction, the amount of residual alcohol is generally in the range from about 20% by weight to about 35% by weight with respect to the weight of starting alcohol.

The catalyst used to carry out the interesterification reaction is preferably an alkali base, an alkaline metal alcoholate, an alkali metal or a strong acid. Advantageously, the catalyst is selected from sodium hydroxide, sodium methylate, sodium metal or 4-toluenesulphonic acid.

The interesterification reaction is generally carried out with stirring for about 0.5 hours to about 10 hours, in an inert atmosphere, for example in a nitrogen atmosphere, and at a temperature of at least about 100° C. to at most about 200° C.

Advantageously, eliminating the catalyst from step b), when this is alkaline, is carried out with an excess of about 500% with respect to the stoichiometric quantity of a strong acid such as sulphuric acid or hydrochloric acid in an aqueous solution of at least N to at most 5N required to neutralize the alkaline catalyst, by stirring at ambient temperature for at least about half an hour to at most about one hour. The catalyst neutralization operation is then followed by washes with water using, for each wash carried out with stirring at a temperature in the range from about 80° C. to about 100° C., a quantity of water of at least about 10% by weight to at most about 20% by weight with respect to the weight of the product to be washed. Between two and four washes are generally necessary to reach neutrality. When the catalyst is a strong acid, it is advantageously eliminated by simple water washes. To carry out the washes at a temperature in the range about 80° C. to about 100° C. with stirring, each wash uses a quantity of water of at least about 10% by weight to at most about 20% by weight with respect to the product to be washed. As many washes as are necessary to obtain a wash water with a neutral pH are carried out.

The residual alcohol in the neutralized product from step c) is distilled at an absolute pressure of the order of 10 to 100 Pascals, at a temperature of at least about 65° C. to at most about 230° C., for a period that is generally at least about 4 hours, preferably about 2 hours. Advantageously, said distillation operation is carried out in the presence of a quantity of decolorizing agent such as activated charcoal of at least about 0.1% by weight to at most about 1% by weight of the product to be distilled. After cooling completely, the decolorizing agent is generally separated from the distillation residue by simple filtering.

The product obtained in step d1) or d2) has a wax ester content (expressed as the percentage with respect to the weight of product obtained) in the range about 55% by weight to about 95% by weight, preferably in the range about 66% by weight to about 90% by weight, and in particular in the range about 70% by weight to about 80% by weight.

A non-greasy emollient based on wax-esters that can be obtained by the process described above has the following characteristics:

it can be a liquid, a solid, or have a greasy consistency at 20° C.;

it is perfectly compatible with the epidermis;

it has a dry and silky feel;

it has good spreadability;

it penetrates rapidly into the epidermis;

it has dermatological properties that are identical to those of the starting oil.

More particularly, the wax-esters obtained are constituted by a mixture of:

66% to 95% by weight of wax-esters;

0.1% to 12% by weight of triglycerides;

3% to 20% by weight of diglycerides; and

1.5% to 10% by weight of monoglycerides (the accumulated proportions of the four components representing 100%, apart from the nonsaponifiable matter, these latter generally representing about 0.5% to 1.5% by weight).

The original problem underlying the present invention is the provision of formulations, in particular formulations for cosmetic use, having:

non-greasy emollient properties, i.e., endowed with a rapid cutaneous penetrating ability, and with additional characteristics consisting of a light and unctuous texture and consistency on application and during spreading, cutaneous penetration of the penetration being progressive during spreading; and

biological properties connected to the presence of natural compounds originating from the natural oil of origin such as nonsaponifiable matter and essential fatty acids.

To this end, the present invention provides a formulation comprising non-greasy emollients based on wax-esters the constituents of which have a molecular weight of less than 610 Daltons, preferably less than 450 Daltons, comprising:

esters of fatty acids and a fatty alcohol derived from interesterification by a said fatty alcohol of triglycerides of an oil of natural origin, preferably vegetable origin; and

residual triglycerides, diglycerides and monoglycerides derived from said intereste-rification;

characterized in that it comprises at least two said wax-esters wherein at least one said wax ester is a hydrogenated wax ester comprising:

hydrogenated fatty acid esters derived from interesterification by one said fatty alcohol of triglycerides of an oil of natural origin, preferably vegetable origin, followed by hydrogenation of said esters; and

residual hydrogenated triglycerides, diglycerides and monoglycerides from said interesterification followed by said hydrogenation.

Thus, said formulation can contain:

either at least one first wax-ester, which is a non-hydrogenated wax ester or Cerester® and at least one second wax ester, which is a hydrogenated wax ester or Phytowax®;

or at least one first wax ester, which is a hydrogenated wax ester, and at least one second wax ester, which is a hydrogenated wax ester, the two said hydrogenated wax-esters being different and in particular having different melting points.

The term “hydrogenation” or “hydrogenated” as used here means that in the hydrocarbon chains, the unsaturated C—C bonds are hydrogenated, said chain then containing only saturated C—C bonds, in particular in the hydrocarbon radical corresponding to the decarboxylated residue of said fatty acid.

In an advantageous implementation, the formulation comprises at least two said hydrogenated wax-esters, the melting points of the at least two said hydrogenated wax-esters differing by at least 10° C., and said melting points being in the range 23° C. to 75° C.

In a more particularly advantageous implementation, at least one said wax ester has a melting point of less than 35° C., preferably less than 30° C., and at least one said hydrogenated wax ester has a melting point of more than 40° C., preferably more than 45° C.

More particularly, the formulation of the invention comprises at least one first wax ester, which is a non-hydrogenated wax ester that is liquid at ambient temperature and at least one second wax ester that is a hydrogenated wax ester which is solid at ambient temperature.

More particularly, said esters of fatty acids and a fatty alcohol comprise:

a C1 to C22 alkyl radical, preferably C6 to C18, corresponding to a dehydroxylated residue of said saturated fatty alcohol; and

a C11 to C21 hydrocarbon radical, preferably C15 to C21, corresponding to a decarboxylated residue of said acid.

In a preferred implementation, a said fatty alcohol is used which is a linear C6 to C18 saturated fatty alcohol selected from 1-hexanol, 1-octanol, 1-decanol, 1-dodecanol, 1-tetradecanol, 1-hexadecanol, 1-octadecanol, oleic alcohol, and hexyldecanol.

The fatty alcohols used advantageously originate from the hydrogenolysis of methyl esters of fatty acids obtained by fractional distillation of the hydrolysis products of vegetable oils. These fatty alcohols of vegetable origin are commercially available.

In an advantageous implemention, said hydrogenated wax-esters comprise a mixture of at least two said esters selected from C6 to C22 alkyl stearates, C6 to C22 alkyl palmitates, C6 to C22 alkyl arachidates and C6 to C22 alkyl hydroxystearates.

Stearic acid derives from hydrogenating oleic, linoleic and linolenic fatty acids. Hydrogenation of the gadoleic acid also present in vegetable oils produces arachidic acid. The hydrogenation product of essential fatty acids from olive oil comprises mainly stearic acid (about 85%). Similarly, the hydrogenation product of castor oil fatty acids comprises mainly 12-hydroxystearic acid (about 85%).

In one implementation, the formulation comprises:

at least one said first hydrogenated wax ester in which said hydrogenated esters comprise one said C1 to C8 alkyl radical, preferably C6 to C8 derived from said saturated fatty alcohol; and

at least one said second hydrogenated wax ester in which said hydrogenated esters comprise one said C10 to C22 alkyl radical derived from said saturated fatty alcohol.

In one particular implementation, the formulation comprises:

at least one said first non-hydrogenated wax ester that is liquid at ambient temperature, in which said non-hydrogenated esters comprise a C1 to C10 alkyl radical, preferably C6 to C10; and

at least one said second hydrogenated wax ester in which said esters comprise one said C6 to C10 alkyl radical.

Advantageously, said saturated fatty acid used to prepare said esters is identical in each of said esters deriving from the same said oil of natural origin, and is different for different wax-esters of the formulation, i.e., those deriving from different oils.

As mentioned above, said wax-esters comprise residual triglycerides, diglycerides and monoglycerides and nonsaponifiable matter deriving from said oil of natural origin in the following respective proportions:

66% to 95% by weight of said optionally hydrogenated esters;

0.1% to 12% by weight of said optionally hydrogenated triglycerides;

3% to 20% by weight of said optionally hydrogenated diglycerides;

1.5% to 10% by weight of said optionally hydrogenated monoglycerides;

0.5% to 1.5% by weight of said optionally hydrogenated nonsaponifiable matter;

the sum of the percentages of these constituents representing 100%.

Said wax-esters used in the formulation of the invention can in particular derive from olive oil, castor oil, sweet almond oil, hazelnut oil, apricot oil, borage oil, rapeseed oil, soybean oil or sunflower seed oil.

In an advantageous implementation of the formulation of the invention, it comprises at least 3, preferably at least 5 said hydrogenated wax-esters.

Said wax-esters are appropriately comprised in the oily phase of an emulsion.

A formulation in accordance with the invention can in particular comprise natural waxes of the beeswax, carnauba wax, candellila wax, ozokerite or ceresin wax type.

A formulation in accordance with the present invention can comprise 0.5% to 15% by weight of said wax-esters.

The present invention also concerns a cosmetic formulation for use in particular as a skin care cream, a foundation cream, an after-shampoo lotion or a lip colouring coating, characterized in that it comprises a formulation in accordance with the invention.

The advantage of the formulations of the present invention resides in the fact that it has a structure and a body, while providing softness, melting properties and unctuousness on application.

Said wax-esters with a melting point of less than about 30° C., i.e., lower than the temperature of the body, impart to the formulation unctuous spreading properties without a greasy feel, and also a cooling sensation on spreading.

Said hydrogenated wax-esters with a melting point that is higher than the temperature of the body impart to the formulation a thickening effect without a greasy feel on spreading while preserving a light unctuous texture. They advantageously replace all of the fatty alcohols conventionally used to provide the formulations with viscosity (C14, C16 or C18 fatty alcohols), and they also limit foaming.

The use of hydrogenated wax-esters in the formulations of the invention impart an agreeable sensorial impact due to the impression of using creams that melt on the skin. The combined use of a plurality of wax-esters reinforces this impression and prolongs the effect.

Further, the process for manufacturing said wax-esters of vegetable origin of the invention that are liquid at ambient temperature preserves the essential fatty acids which have active properties that are already well known. They can thus combine the advantages of essential fatty acids without the disadvantage of a non-greasy feel.

The process of the present invention can use any existing vegetable oil, thereby profiting from the wide diversity of compositions containing the fatty acids of those oils, and also from the nonsaponifiable matter in the starting oil.

Depending on the botanical origin of the oil involved in the production of Ceresters®, these latter may contain greater or lesser amounts of essential fatty acids such as linoleic acid, the role of which is well known in limiting transepidermal water loss. This is the case with sunflower Ceresters®, which supply of the order of 60% of the linoleic acid, without the disadvantage of the greasy feel associated with the original sunflower oil.

Further, depending on the degree of alcoholysis of the triglycerides in the oil employed, the wax-esters still contain a greater or lesser proportion of residual monoglycerides which endow these products with an emulsifying effect.

Other characteristics and advantages of the present invention will become apparent from the illustrative examples detailed below.

Completely hydrogenated wax-esters were prepared from olive oil, more particularly a range of 7 products was prepared with melting points that ranged regularly from 25° C. to 57° C. This range was obtained by alcoholysis of olive oil (C18 fatty acids) with different fatty alcohols of vegetable origin with a carbon condensation between 6 and 18. A range of products with a completely vegetable origin was obtained with molecular weights of between 366 and 534 Daltons.

The hydrogenated wax-esters prepared were solids at 20° C., in which all of the essential fatty acids had been hydrogenated to stearic acid. Since olive oil is constituted by 85% C18 acids and 14% C16 acids, the wax-esters derived from its hydrogenation were constituted by 85% alkyl stearate with a melting point that increased with the carbon condensation of the alcohol employed to produce the wax-esters. It was then possible to constitute a range of products with increasing melting points by changing the length of the alcohol. By carrying out the same procedure with castor oil constituted by 85% ricinoleic acid (monounsaturated), hydrogenation produced a product constituted by 85% hydroxystearic acid (saturated) which enabled the “olive” range to be completed by products with higher melting points. A range of completely saturated wax-esters of vegetable origin was thus obtained, with various melting points which, when used individually or as a mixture, provide a number of feel sensations that are novel in cosmetics.

To make up wax ester formulations that are liquid at ambient temperature, we selected olive Ceresters® because of their relative stability to oxidation, and because of the media image of this oil. We also used sweet almond oil Ceresters® because of the universal use of sweet almond oil in cosmetics. Finally, we selected sunflower Ceresters® for the high percentage of linoleic acid in sunflower oil, an acid considered to be essential and which, when incorporated into ceramides, can limit percutaneous water loss.

A range of 10 solid wax-esters obtained from olive oil and castor oil were tested individually or in combined form:

| | | | | | | Saponifi- | Melting |
|---|---|---|---|---|---|---|---|
| | Appear- | | | Iodine | Acid- | cation | point |
| “Phytowax” | ance | Colour | Alcohol | No. | ity | value | (° C.) |
| OLIVE 6L 25 | Paste | White | 1-hexanol | <4 | <2 | 120-150 | 23-28 |
| OLIVE 8L 25 | Solid | White | 1-octanol | <4 | <2 | 120-150 | 27-32 |
| OLIVE 10 | Solid | White | 1-decanol | <4 | <2 | 120-150 | 35-42 |
| 40 | | | | | | | |
| OLIVE 12 | Solid | White | 1-dodecanol | <4 | <2 | 120-150 | 40-45 |
| 44 | | | | | | | |
| OLIVE 14 | Solid | White | 1-tetradecanol | <4 | <2 | 120-150 | 45-52 |
| 48 | | | | | | | |
| OLIVE 16 | Solid | White | 1-hexadecanol | <4 | <2 | 100-140 | 52-57 |
| 55 | | | | | | | |
| OLIVE 18 | Solid | White | 1-octadecanol | <4 | <2 | 100-140 | 54-60 |
| 57 | | | | | | | |
| CASTOR 16 | Solid | Yellowish | 1-hexadecanol | <4 | <2 | 100-140 | 62-66 |
| 64 | | | | | | | |
| CASTOR 18 | Solid | Yellowish | 1-octadecanol | <4 | <2 | 100-140 | 67-71 |
| 69 | | | | | | | |
| CASTOR 22 | Solid | Yellowish | 1-docosanol | <4 | <2 | 90-120 | 71-75 |
| 73 | | | | | | | |

The figures shown in the definition of the Phytowax products, for example 6L 25, mean the following:

the first figure represents the number of fatty alcohol carbons used, 6 in the selected example;

the letter L represents the fact that the alcohol is linear;

and the second figure shows the mean melting point of the product, 25° C. for the selected example.

in the above table:

the iodine number is defined as the mass in grams (g) of iodine fixed by 100 g of sample (French standard NF ISO 3961). Each double bond fixes one mole of iodine (I₂). The value 4 given for the iodine number is an upper limit fixed by the specifications and is not an actual measurement;

the acidity referred to does not correspond to a pH but to an acidity expressed as the acid value, defined as the number of milligrams of potassium hydroxide required to neutralise the free fatty acids in 1 g of fat (French standard NF T 60.204);

the saponification value is defined as the number of milligrams of potassium hydroxide required to saponify 1 g of fat (International standard ISO 3657: 1988 F).

### EXAMPLE 1

### Protocol for Preparing Olive 10L 40Phytowax®

1.1 670 g of refined olive oil was placed in a single-necked flask. 330 g of 1-decanol in which 0.5 g of sodium had been dissolved was added. After forming a 5000 pascal (Pa) absolute vacuum, the temperature was raised to 125° C. On reaching that temperature, the atmosphere of the flask was slightly pressurized with nitrogen. After stirring for 30 minutes at 125° C., the interesterification reaction had reached the desired level.

1.2 The product obtained above underwent the following treatment. The product was maintained under vacuum in the reaction flask, then 50 ml of an aqueous 2N sulphuric acid solution was added. The temperature was raised to 90° C., it was stirred for 15 minutes and allowed to settle. The aqueous acid phase was extracted, 100 ml of water was added, it was stirred for 10 minutes at 90° C., then allowed to settle. This water wash was repeated twice to reach neutrality. The product was allowed to settle completely and then completely dried under reduced pressure at 95° C. 960 g of product was recovered, to which 2.4 g of activated charcoal was added. The mixture was vacuum distilled (70 Pa) with nitrogen microbubbling, gradually heating the flask ensuring that the temperature reached by the fluid at the end of the distillation did not exceed 180° C. The vacuum during distillation was about 60 Pa. Distillation was halted after 2 hours. 900 g of product was recovered in the flask and filtered over paper to separate the activated charcoal. 880 g of a yellow liquid product was obtained in which a slight precipitate appeared.

1.3 The product obtained above was hydrogenated in a stirred reactor with 1% of nickel-based catalyst deposited on silica (25% of nickel in the catalyst) at a pressure of 10 bars of hydrogen, at 200° C., for 6 hours. After filtering off the catalyst, an off-white coloured product was obtained with a melting point of 40° C. and with an iodine number of less than 1.

The product obtained after hydrogenation and filtration comprised (per 100 grams of product obtained:

82.0 grams of wax-esters, constituted by:

69.7 g of decyl octadecanoate (decyl stearate), MW=424 Daltons;

11.9 g of decyl hexadecanoate (decyl palmitate);

0.4 g of decyl eicosanoate (decyl arachidate);

5.5 grams of triglycerides (14.5% palmitic, 85.0% stearic, 0.5% arachidic);

6.1 grams of diglycerides (14.5% palmitic, 85.0% stearic, 0.5% arachidic);

4.1 grams of monoglycerides (14.5% palmitic, 85.0% stearic, 0.5% arachidic).

### EXAMPLE 2

### Protocol for Preparing Olive 10 Cerester®

875 g of the product obtained at 1.2 above (Example 1) was introduced into a cylindrical reactor equipped with an external jacket for the passage of a coolant. The liquid was gradually cooled to a temperature of 14.5° C. by passing the coolant through the jacket of said wintering reactor at 14° C., with stirring for 4 hours, then it was filtered. 850 g of a yellow coloured liquid was obtained which had no marked odor and which was perfectly clear at 15° C.

The product obtained after wintering and filtering comprised (per 100 grams of product obtained):

82.2 grams of wax ester, constituted by:

60.9 g of 1-decyl 9-octadecene (decyl stearate), MW=422 Daltons;

10.2 g of 1-decyl hexadecanoate (decyl palmitate);

1.8 g of 1-decyl 9-hexadecene (decyl palmitoleate);

2.0 g of 1-decyl octadecanoate (decyl stearate);

6.4 g of 1-decyl 9,10-octadecadiene (decyl linoleate);

0.5 g of 1-decyl 9, 10, 12-octadecatriene (decyl α-linolenate);

0.1 g of decyl eicosanoate (decyl arachidate);

0.3 g of decyl 11-eicosene (decyl gadoleate);

5.5 grams of triglycerides;

6.0 grams of diglycerides;

4.0 grams of monoglycerides.

### EXAMPLE 3

### Protocol for Preparing Olive 6 L25 Phytowax®

750 g of olive oil was placed in a single-necked flask and 242 g of hexanol in which 0.5 g of sodium had been dissolved was added. After forming a vacuum of 10000 Pa absolute, the temperature was raised to 100° C. On reaching that temperature, the vacuum was broken and the atmosphere of the flask was slightly pressurized with nitrogen. The temperature was then raised to 125° C. and maintained at that temperature for 30 minutes. The flask was then cooled to ambient temperature. The product obtained underwent the same catalyst elimination treatment by washing with 2N sulphuric acid as the product in Example 1.2. After eliminating the washing water, 0.25% by weight of charcoal was added to the product obtained. The product was then distilled at 180° C. under a 60 Pa vacuum for 2 hours. After cooling, the product was filtered through paper. The filtrate obtained was then hydrogenated under the same conditions as described in Example 1.3. After filtering the catalyst, a product was obtained with a melting point of 25.0° C. and with an iodine number of less than 1.

The product obtained after hydrogenation and filtering comprised (per 100 grams of product):

89.1 grams of wax-esters, constituted by:

74.9 g of 1-hexyl octadecanoate (hexyl stearate), MW=368 Daltons;

13.1 g of 1-hexyl hexadecanoate (hexyl palmitate);

1.1 g of 1-hexyl eicosanoate (hexyl arachidate);

1.1 grams of triglycerides (14.5% palmitic, 85.0% stearic, 0.5% arachidic);

3.4 grams of diglycerides (14.5% palmitic, 85.0% stearic, 0.5% arachidic).

### EXAMPLE 4

### Protocol for Preparing Olive 8 L28 Phytowax®

700 g of refined olive oil was placed in a single-necked flask. 300 g of 1-octanol in which 0.55 g of sodium had been dissolved was added. After forming a vacuum of 5000 Pa absolute, the temperature was raised to 100° C. On reaching that temperature, the vacuum was broken and the atmosphere of the flask was slightly pressurized with nitrogen. The temperature was then raised to 125° C. and kept at that level for 45 minutes. The flask was then cooled to ambient temperature. After treating the product with 50 ml of 2N sulphuric acid at 90° C. with stirring for 15 minutes, it was allowed to settle. After extracting the aqueous phase and washing with water to neutrality, the product was dried under reduced pressure at 95° C. After adding 0.25% of activated charcoal, the residual 1-octanol was distilled off under vacuum (70 Pa). Distillation was stopped after 2 hours. After cooling, the product was filtered through paper, the filtrate was hydrogenated in a stirred reactor with 1% of nickel-based catalyst deposited on silica (25% of nickel in the catalyst) at a pressure of 10 bars of hydrogen, at 200° C., for 6 hours. After filtering off the catalyst, a whitish product was obtained with a melting point of 29° C.

The product obtained after hydrogenation and filtration comprised, per 100 grams of product obtained:

82.5 grams of wax-esters, constituted by:

71.1 g of octyl octadecanoate (caprilyl stearate);

10.1 g of octyl hexadecanoate (caprilyl palmitate);

0.4 g of octyl eicosanoate (caprilyl arachidate);

7.4 grams of triglycerides (14.5% palmitic, 85.0% stearic, 0.5% arachidic);

3.6 grams of diglycerides (14.5% palmitic, 85.0% stearic, 0.5% arachidic);

4.1 grams of monoglycerides (14.5% palmitic, 85.0% stearic, 0.5% arachidic).

### EXAMPLE 5

### Protocol for Preparing Olive 14 L48 Phytowax®

600 g of olive oil was placed in a single-necked flask. 402 g of 1-tetradecanol in which 0.8 g of sodium had been dissolved was added. After forming a vacuum of 5000 Pa absolute, the temperature was raised to 100° C. On reaching that temperature, the vacuum was broken and the atmosphere of the flask was slightly pressurized with nitrogen. The temperature was then raised to 125° C. and kept at that level for 30 minutes. The flask was then cooled to ambient temperature. The product obtained underwent the same catalyst elimination treatment by washing with 2N sulphuric acid as the product in Example 1.2. After eliminating the washing water, 0.25% by weight of charcoal was added to the product obtained. The product was then distilled at 180° C. under a 60 Pa vacuum for 2 hours. After cooling, the product was filtered through paper. The filtrate obtained was then hydrogenated under the same conditions as described in Example 1.3. After filtering the catalyst, a product was obtained with a melting point of 48° C. and with an iodine number of less than 1.

The product obtained after hydrogenation and filtration comprised, per 100 grams of product obtained:

78.9 grams of wax-esters, constituted by:

64.0 g of 1-tetradecyl octadecanoate (myristyl stearate), MW=480 Daltons;

13.6 g of 1-tetradecyl hexadecanoate (myristyl palmitate);

1.3 g of 1-tetradecyl eicosanoate (myristyl arachidate);

8.8 grams of triglycerides (14.5% palmitic, 85.0% stearic, 0.5% arachidic);

5.3 grams of diglycerides (14.5% palmitic, 85.0% stearic, 0.5% arachidic);

3.9 grams of monoglycerides (14.5% palmitic, 85.0% stearic, 0.5% arachidic).

### EXAMPLE 6

### Protocol for Preparing Olive 16 L55 Phytowax®

565 g of olive oil was placed in a single-necked flask. 435 g of 1-hexadecanol in which 1.5 g of sodium had been dissolved was added. After forming a vacuum of 5000 Pa absolute, the temperature was raised to 125° C. On reaching that temperature, the atmosphere of the flask was slightly pressurized with nitrogen. After 1 hour's reaction at 125° C., the flask was cooled. After treating the product with 50 ml of 2N sulphuric acid at 90° C. with stirring for 15 minutes, and allowed to settle. After extracting the aqueous phase and washing with water to neutrality, the product was dried under reduced pressure at 95° C. After adding 0.25% of activated charcoal, the residual 1-hexadacanol was distilled off under vacuum (50 Pa). Distillation was stopped after 2 hours. The product was then hot filtered through paper under a nitrogen atmosphere. The filtrate was hydrogenated in a stirred reactor with 1% of nickel-based catalyst deposited on silica (25% of nickel in the catalyst) at a pressure of 10 bars of hydrogen, at 200° C., for 6 hours. After filtering off the catalyst, a whitish product was obtained with a melting point of 56° C.

The product obtained after hydrogenation and filtration comprised, per 100 grams of product obtained:

80.1 grams of wax-esters, constituted by:

67.5 g of hexadecyl octadecanoate (palmityl stearate), MW=508 Daltons;

10.6 g of hexadecyl hexadecanoate (palmityl palmitate);

0.4 g of hexadecyl eicosanoate (palmityl arachidate);

11.5 grams of triglycerides (14.5% palmitic, 85.0% stearic, 0.5% arachidic);

3.9 grams of diglycerides (14.5% palmitic, 85.0% stearic, 0.5% arachidic);

2.1 grams of monoglycerides (14.5% palmitic, 85.0% stearic, 0.5% arachidic).

### EXAMPLE 7

### Protocol for Preparing Olive 18 L57 Phytowax®

541 g of refined olive oil was placed in a single-necked flask. 456 g of 1-octadecanol in which 0.7 g of sodium had been dissolved was added. After forming a vacuum of 5000 Pa absolute, the temperature was raised to 125° C. and the contents of the flask were stirred to homogenize the reaction medium. On reaching that temperature, the vacuum was broken and the reaction medium was pressurized with atmospheric nitrogen. After 6 hours of reaction at 180° C., the flask was cooled.

The product obtained underwent the same catalyst elimination treatment by washing with 2N sulphuric acid as the product in Example 1.2. After eliminating the washing water, 0.25% by weight of charcoal was added to the product obtained. The product was then distilled at 230° C. under a 50 Pa vacuum for 2 hours. After cooling to 60° C., the product was filtered through paper at that temperature to eliminate the decolorizing agent. The filtrate obtained was then hydrogenated under the same conditions as described in Example 1.3. After filtering the catalyst, a product was obtained with a melting point of 57° C. and with an iodine number of less than 1.

The product obtained after hydrogenation and filtration comprised (per 100 grams of product obtained):

78.0 grams of wax-esters, constituted by:

66.3 g of 1-octadecyl octadecanoate (stearyl stearate), MW=536 Daltons;

11.3 g of 1-octadecyl hexadecanoate (stearyl palmitate);

0.4 g of 1-octadecyl eicosanoate (stearyl arachidate);

6.9 grams of triglycerides (14.5% palmitic, 85.0% stearic, 0.5% arachidic);

5.0 grams of diglycerides (14.5% palmitic, 85.0% stearic, 0.5% arachidic);

7.6 grams of monoglycerides (14.5% palmitic, 85.0% stearic, 0.5% arachidic).

### EXAMPLE 8

### Protocol for Preparing Castor 16 L64 Phytowax®

567 g of hydrogenated castor oil was placed in a single-necked flask, and then 433 g of 1-hexadecanol in which 1.5 g of sodium had been dissolved was added. After forming a vacuum of 5000 Pa absolute, the temperature was raised to 125° C. On reaching that temperature, the atmosphere of the flask was slightly pressurized with nitrogen. After reacting for 1 hour at 125° C., it was cooled. After treating the product with 50 ml of 2N sulphuric acid at 90° C. with stirring for 15 minutes, it was allowed to settle. After extracting the aqueous phase and washing with water to neutrality, the product was dried under reduced pressure at 95° C. After adding 0.25% of activated charcoal, the residual 1-hexadecanol was distilled off under vacuum (50 Pa). Distillation was stopped after 2 hours. The product was then hot filtered through paper under a nitrogen atmosphere. A yellowish product was obtained with a melting point of 64° C.

The product obtained comprised, per 100 g:

61.0 grams of wax-esters, constituted by:

51.4 g of hexadecyl 12-hydroxyoctadecanoate (palmityl hydroxystearate);

8.5 g of hexadecyl octadecanoate (palmityl stearate);

1.3 g of hexadecyl hexadecanoate (palmityl palmitate);

9.8 grams of triglycerides (1.8% palmitic, 13.8% stearic, 84.3% hydroxystearic);

2.1 grams of monoglycerides (1.8% palmitic, 13.8% stearic, 84.3% hydroxystearic);

23.1% of estolides (hydroxystearic acid polyesters).

### EXAMPLE 9

### Protocol for Preparing Castor 18 L69 Phytowax®

540 g of hydrogenated castor oil was placed in a single-necked flask, and then 460 g of 1-octadecanol in which 1.5 g of sodium had been dissolved was added. After forming a vacuum of 5000 Pa absolute, the temperature was raised to 125° C. On reaching that temperature, the atmosphere of the flask was slightly pressurized with nitrogen. After reacting for 1 hour at 125° C., it was cooled. After treating the product with 50 ml of 2N sulphuric acid at 90° C. with stirring for 15 minutes, it was allowed to settle. After extracting the aqueous phase and washing with water to neutrality, the product was dried under reduced pressure at 95° C. After adding 0.25% of activated charcoal, the residual 1-octadecanol was distilled off under vacuum (50 Pa). Distillation was stopped after 2 hours. The product was then hot filtered through paper under a nitrogen atmosphere. A yellowish product was obtained with a melting point of 69° C.

The product obtained comprised, per 100 g:

63.4 grams of wax-esters, constituted by:

53.3 g of octadecyl 12-hydroxyoctadecanoate (stearyl hydroxystearate);

9.0 g of octadecyl octadecanoate (stearyl stearate);

1.1 g of octadecyl hexadecanoate (stearyl palmitate);

5.2 grams of triglycerides (1.8% palmitic, 13.8% stearic, 84.3% hydroxystearic);

2.2 grams of diglycerides (1.8% palmitic, 13.8% stearic, 84.3% hydroxystearic);

1.3 grams of monoglycerides (1.8% palmitic, 13.8% stearic, 84.3% hydroxystearic);

26.0% of estolides (hydroxystearic acid polyesters).

### EXAMPLE 10

### Protocol for Preparing Castor 22 L73 Phytowax®

493 g of hydrogenated castor oil was placed in a single-necked flask, and then 507 g of 1-docosanol in which 1.5 g of sodium had been dissolved was added. After forming a vacuum of 5000 Pa absolute, the temperature was raised to 125° C. On reaching that temperature, the atmosphere of the flask was slightly pressurized with nitrogen. After reacting for 1 hour at 125° C., it was cooled. After treating the product with 50 ml of 2N sulphuric acid at 90° C. with stirring for 15 minutes, it was allowed to settle. After extracting the aqueous phase and washing with water to neutrality, the product was dried under reduced pressure at 95° C. After adding 0.25% of activated charcoal, the residual 1-docosanol was distilled off under vacuum (50 Pa). Distillation was stopped after 2 hours. The product was then hot filtered through paper under a nitrogen atmosphere. A yellowish product was obtained with a melting point of 73° C.

The product obtained comprised, per 100 g:

48.5 grams of wax-esters, constituted by:

36.2 g of docosanyl 12-hydroxyoctadecanoate (behenyl hydroxystearate);

11.2 g of docosanyl octadecanoate (behenyl stearate);

1.1 g of docosanyl hexadecanoate (behenyl palmitate);

11.9 grams of triglycerides (1.8% palmitic, 13.8% stearic, 84.3% hydroxystearic);

5.2 grams of diglycerides (1.8% palmitic, 13.8% stearic, 84.3% hydroxystearic);

1.9 grams of monoglycerides (1.8% palmitic, 13.8% stearic, 84.3% hydroxystearic);

30.0% of estolides (hydroxystearic acid polyesters).

### EXAMPLE 11

### Interesterification Using Sweet Almond Oil Sunflower Oil, Rapeseed Oil and Hazelnut Oil

11.1 709 g of refined sweet almond oil was placed in a single-necked flask, then 291 g of 1-octanol and 1.4 g of sodium methylate were added. After forming a vacuum of 5000 Pa absolute, the temperature was raised to 100° C. On reaching that temperature, the vacuum was broken and the atmosphere of the flask was slightly pressurised with nitrogen. The temperature of the flask was then raised to 170° C. and kept at that temperature for 6 hours. The flask was then cooled to ambient temperature.

11.2 670 g of oleic sunflower oil was placed in a single-necked flask, and then 330 g of 1-decanol and 1.5 g of 4-toluenesulphonic acid were added. After forming a vacuum of 5000 Pa absolute, the temperature was raised to 150° C. On reaching that temperature, the atmosphere of the flask was slightly pressurized with nitrogen. After 6 hours of stirring, the interesterification reaction had reached the desired level.

11.3 670 g of completely hydrogenated refined rapeseed oil was placed in a single-necked flask, and then 330 g of 1-decanol in which 0.5 g of sodium had been dissolved was added. After forming a vacuum of 5000 Pa absolute, the temperature was raised to 125° C. On reaching that temperature, the atmosphere of the flask was slightly pressurized with nitrogen. After stirring for 30 minutes at 125° C., the inter-esterifiation reaction had reached the desired level.

11.4 670 g of refined hazelnut oil was placed in a single-necked flask, and then 330 g of 1-decanol in which 0.5 g of sodium had been dissolved was added. After forming a vacuum of 5000 Pa absolute, the temperature was raised to 125° C. On reaching that temperature, the atmosphere of the flask was slightly pressurized with nitrogen. After stirring for 30 minutes at 125° C., the interesterification reaction had reached the desired level.

11.5 The products obtained above in 11.1 to 11.4 underwent the wintering treatment described in Example 2 to obtain the corresponding Ceresters®.

EXAMPLE 12
Skin care cream formulation

| Phase | Starting materials | % w/w |
|---|---|---|
| A | Cetearyl glucosinate and cetearyl alcohol | 6.000 |
| | Hydrogenated polyisobutene | 10.000 |
| | Paraffin | 2.000 |
| | Olive 10 Cerester ® | 4.000 |
| | Olive 6L 25 Phytowax ® | 3.000 |
| | Squalane | 5.000 |
| | Sitosterol | 0.5000 |
| | Antioxidant | q.s. |
| B | Water | q.s.p. |
| | Glycerol | 2.000 |
| | Preservatives | q.s. |
| C | Copolymer of acrylamide and mineral oil and | 1.000 |
| | C₁₃-C₁₄ isoparaffin and Polysorbate 85 | |
| D | Triethanolamine | 0.025 |
| E | Fragrance | q.s. |

Manufacturing procedure:
1) Heat A to 80° C.;
2) Heat B to 80° C.;
3) Pour 2) into 1) with stirring;
4) Emulsify;
5) Add C without stirring, at 50° C.;
6) Add D;
7) Add E at 30° C.

EXAMPLE 13
Night cream formulation

| Phase | | % w/w |
|---|---|---|
| A | PEG 8 beeswax | 10.000 |
| | Hydrogenated polyisobutene | 11.000 |
| | Sunflower 10 Cerester ® | 15.000 |
| | Olive 6L 25 Phytowax ® | 3.500 |
| | PEG 100 stearate and glyceryl stearate | 5.000 |
| | Preservative | q.s. |
| B | Water | q.s.p. 100 |
| | Preservative | q.s |
| | Hydroxy Ethyl Cellulose | 0.400 |
| C | Triethanolamine | 0.400 |
| D | Water | 20.000 |
| | Carbomer 934 | 0.400 |
| E | Fragrance | q.s. |

Manufacturing procedure:
1) Heat water in a bath to 70° C.; dust on the carbomer with vigorous stirring;
2) Heat A to 80° C.;
3) Heat B to 80° C. with vigorous stirring;
4) Pour 2) into 3) with stirring;
5) Add C to 1) with stirring;
6) Add 5) to 4) with stirring;
7) At 35° C., add E with stirring.

EXAMPLE 14
Skin care cream formulation

| | | % w/w |
|---|---|---|
| A | Water | 64.300 |
| B | Preservatives | q.s. |
| | Glycerine | 3.000 |
| C | Cetearyl glucoside | 5.000 |
| | Cetearyl alcohol | |
| | Caprylic alcohol triglyceride | 5.000 |
| | Nut butter | 1.000 |
| | Olive 10 Cerester ® and | 3.000 |
| | squalene | |
| | Olive squalane | 5.000 |
| | Olive 6L 25 Phytowax ® | 2.000 |
| | Olive 16L 55 Phytowax ® | 3.000 |
| | Olive 18L 57Phytowax ® | 3.000 |
| D | Tocopherol acetate | 0.200 |
| E | Methacrylate copolymer | 3.000 |
| F | Cyclomethicone | 1.000 |
| G | Polyacrylamide - C₁₃-C₁₄ isopa- | 1.000 |
| | raffin - Laureth-7 | |
| H | Fragrances | q.s. |

The operating procedure is conventional:
1) Introduce A into the stainless steel bath equipped with a turbo-emulsifier and an anchor agitator;
2) Heat to 75° C.;
3) Add B, previously hot homogenized;
4) Pre-melt fat phase C;
5) Add fat phase C to bath at 75° C. and stir for 15 minutes, stirring with the emulsifier and anchor agitator;
6) Cool slowly to 45° C.;
7) Then add D;
8) Add E then F;
9) Add H, cold;
10) Add G.

This cream using 3 Phytowax® products had a texture that combined unctuousness, softness and ease of application.

The different tests carried out to produce such a formula showed that:

If a C10 Phytowax® product has a melting point close to the skin temperature, it is still too high to melt on the skin and thus tends to produce lumps during application and encourage the formation of flakes.

Phytowax® products with a melting point lower than the body temperature impart to the cosmetic formulation an unctuous spread without a greasy feel, and when a cooling sensation on spreading. The C6 Phytowax® product in particular is particularly advantageous as it imparts a very characteristic softness on spreading without a greasy effect.

Phytowax® products with a melting point higher than the body temperature impart to the cosmetic formulation a thickening effect without a greasy feel on spreading. Thus, 16 L55 and 18 L57 Phytowaxes® are more advantageous for producing structure at ambient temperature while preserving a light and unctuous texture.

The tests also showed that Phytowax® products can advantageously replace all of the currently used fatty alcohols to endow the formulations with viscosity (C14, C16 or C18 fatty alcohols) and they also limit the soap effect, i.e., the appearance of white traces on spreading a cream.

EXAMPLE 15
Liquid foundation cream formulation

| Phase | | % w/w |
|---|---|---|
| A | Water | 54.000 |
| | Xanthan gum | 0.150 |
| | Preservatives | q.s. |
| B | PEG 100 stearate - glyceryl | 4.000 |
| | Stearate | |
| | Olive squalane | 11.000 |
| | Olive 6L 25 Phytowax ® | 3.000 |
| | Olive 16L 55 Phytowax ® | 5.000 |
| C | Isodecyl isonanoate | 6.000 |
| | Titanium dioxide | q.s. |
| D | Tocopherol acetate | 0.200 |
| E | Polymethyl methacrylate | 3.000 |
| F | Fragrance | q.s. |
| G | C₁₃-C₁₄ iso-paraffin | 1.000 |
| | polyacrylamide | |

This composition involves two Phytowax® products to produce a foundation that spreads very well and has a non-greasy texture:

6L 25 Phytowax® imparts spreadability with the impression of the cream melting on the skin.

16L 55 Phytowax® thickens the cream and provides texture without a residual greasy feel and without a soap effect.

EXAMPLE 16
Cream formulation for compact
foundation

| Phase | | % w/w |
|---|---|---|
| A | Olive squalene | 21.700 |
| | Olive Cerester ® | 1.000 |
| | Preservatives | q.s. |
| B | Olive 6L 25 Phytowax ® | 6.000 |
| | Olive 8L 28 Phytowax ® | 1.000 |
| | Beeswax | 2.000 |
| | Ozokerite | 5.000 |
| C | Hydroxystearic acid | 2.000 |
| D | Hydrogenated polyisobutene | 8.000 |
| | Titanium dioxide | q.s. |
| | Pigments | q.s. |
| E | Methacrylate copolymer | 20.000 |
| | Aluminium octenylsuccinate starch | 10.000 |
| | Nylon-12 | 3.000 |
| | Corn starch | 7.000 |
| F | Fragrances | q.s. |

This foundation using one Cerester® and two Phytowax® products is very unctuous to the touch and provides a very characteristic powdery effect on application.

EXAMPLE 17
After-shampoo hair care formulation:

| | % w/w |
|---|---|
| Water | 87.000 |
| Preservatives | q.s. |
| Ceteraryl glucosinate and | 5.000 |
| cetearyl alcohol | |
| Stearalkonium chloride | 2.000 |
| Olive 6L 28 Phytowax ® | 1.000 |
| Olive 12L 44 Phytowax ® | 1.500 |
| Glycerol | 2.00 |
| Polyquaternium-7 | 0.50 |
| Panthenol | 0.50 |
| Tocopheryl acetate | 0.250 |
| Fragrance | 0.20 |

This cream using 2 Phytowax® products, C10 and C12, imparts a melting effect on application.

It has also been shown that a C10 or C12 Phytowax® encourages coating of the hair.

EXAMPLE 18
Lipstick formulation

| | | % w/w |
|---|---|---|
| A | Castor oil | 38.200 |
| | Squalane | 10.000 |
| | Olive 6L 25 Phytowax ® | 5.000 |
| | Olive 10 Cerester ® | 2.000 |
| B | Beeswax | 10.000 |
| | Ozokerite | 8.000 |
| | Olive 6L 25 Phytowax ® | 1.000 |
| | Olive 8L 28 Phytowax ® | 3.000 |
| | Olive 10L 40 Phytowax ® | 4.000 |
| | Olive 12L 44 Phytowax ® | 3.000 |
| | Olive 16L 55 Phytowax ® | 4.000 |
| | Preservatives | q.s. |
| C | Isodecyl isonanoate or isononyl | 3.000 |
| | isononanoate | |
| | Pigments | q.s. |
| D | Methacrylate copolymer | 2.000 |
| | Nylon-12 | 1.000 |
| E | Tocopherol acetate | 0.200 |
| F | Fragrance | q.s. |

One Cerester® and 5 different Phytowax® products are used in the composition of this lipstick formulation. Using the Phytowax® imparts structure to the stick and improves the crystalline matrix.

The lipstick is soft and unctuous on application. The Phytowax® products improve unmoulding on production.

A number of variations of this lipstick formula were produced:

A first test consisted of substituting the castor oil with a castor Cerester®: the test showed a substantial improvement in the sensorial profile of the lipstick as regards spreading, slide on application, and better stability at 500 after 24 hours compared with the control.

Similarly, a test using olive Cerester® as a substitute for the isononyl isononanoate had very good pigment mass dispersion properties.

This ability to disperse pigments was then specifically tested:

a test with organophilic TiO₂ showed:

with castor oil, a dispersion of 60%;

with a castor Cerester®, a dispersion of 75%;

a test with an organic pigment (DC Red7) showed:

with castor oil, a dispersion of 20%;

with a castor Cerester®, a dispersion of 28%.

Regarding the structure of the crystalline matrix, it was noticed that unsatisfactory results were obtained when the beeswax and ozokerite in the lipstick formula were completely substituted; the lipstick did not have sufficient structure to be unmoulded.

Thus, 10% of the beeswax used in the basic formulation was replaced with 10% of castor Phytowax® distributed between 16 L64, 18 L69 and 22 L73. This test provided consistency and body equivalent to that of the basic formula with beeswax.

However in particular, this test showed that the stability to heat of this sample was better; the test sample containing Phytowax® exuded less at 42° C. and 50° C. than the control.

Buoyed up by this result, tests wherein the waxes were partially replaced with castor 22 L73 Phytowax® were carried out. It appears that a replacement of the order of 30% of the traditional waxes by castor 22 L73 Phytowax® had the effect of obtaining an improvement of the order of 20% in the strength of the lipstick and thus in the crystalline structure. This property means that the quantity of wax used can be reduced, or an equal quantity would improve the strength of the lipstick.

The Phytowax® products with a melting point that is lower than body temperature used in the formulations have confirmed their positive effect on the spreadability of the lipstick, which is unctuous without a greasy feel, spreadability being facilitated by the mixture of said Phytowax® products.

1. A formulation comprising non-greasy emollients based on wax-esters the constituents of which have a molecular weight of less than 600 Daltons, preferably less than 450 Daltons, comprising:
esters of fatty acids and a fatty alcohol deriving from interesterification by a said fatty alcohol of triglycerides of an oil of natural origin, preferably vegetable origin; and
residual triglycerides, diglycerides and monoglycerides deriving from said interesterification; characterized in that it comprises at least two said wax-esters wherein at least one said wax ester is a hydrogenated wax ester comprising:
hydrogenated fatty acid esters derived from interesterification by a said fatty alcohol of triglycerides of an oil of natural origin, preferably vegetable origin, followed by hydrogenation of said esters; and
residual hydrogenated triglycerides, diglycerides and monoglycerides from said interesterification, followed by said hydrogenation.

2. A formulation according to claim 1, characterized in that it comprises at least two different said hydrogenated wax-esters.

3. A formulation according to claim 2, characterized in that the melting points of said two hydrogenated wax-esters differ by at least 10° C., and said melting points are in the range 23° C. to 75° C.

4. A formulation according to any one of claims 1 to 3, characterized in that at least one said wax ester has a melting point of less than 35° C., preferably less than 30° C., and at least one said hydrogenated wax ester has a melting point of more than 40° C., preferably more than 45° C.

5. A formulation according to claim 5, characterized in that it comprises at least one first wax ester which is a non-hydrogenated wax ester and at least one second wax ester which is a hydrogenated wax ester.

6. A formulation according to claim 5, characterized in that it comprises at least one first wax ester which is a non-hydrogenated wax ester that is liquid at ambient temperature and at least one second wax ester which is a hydrogenated wax ester that is solid at ambient temperature.

7. A formula according to any one of claims 1 to 6, characterized in that said esters of fatty acids and a fatty alcohol comprise:
a C1 to C22 alkyl radical, preferably C6 to C18, corresponding to a dehydroxylated residue of said saturated fatty alcohol; and
a C11 to C21 hydrocarbon radical, preferably C15 to C21, corresponding to a decarboxylated residue of said acid.

8. A formulation according to claim 7, characterized in that it comprises:
at least one said first hydrogenated wax ester in which said hydrogenated esters comprise a said C1 to C8 alkyl radical, preferably C6 to C8, derived from said saturated fatty alcohol; and
at least one said second hydrogenated wax ester in which said hydrogenated esters comprise a said C10 to C22 alkyl radical derived from said saturated fatty alcohol.

9. A formulation according to claim 7, characterized in that it comprises:
at least one said first non-hydrogenated wax ester that is liquid at ambient temperature, in which said non-hydrogenated esters comprise a C1 to C10 alkyl radical, preferably C6 to C10; and
at least one said second hydrogenated wax ester in which said esters comprise a said C6 to C10 alkyl radical.

10. A formulation according to any one of the preceding claims, characterized in that said wax-esters originate from olive oil, castor oil, sweet almond oil, borage oil, hazelnut oil, sunflower oil, rapeseed oil, soybean oil and apricot oil.

11. A formulation according to any one of the preceding claims, characterized in that said wax-esters comprise a mixture of at least two said esters selected from C6 to C22 alkyl stearates, C6 to C22 alkyl palmitates, C6 to C22 alkyl arachidates, and C6 to C22 alkylhydroxystearates.

12. A formulation according to any one of the preceding claims, characterized in that it comprises at least 3, preferably at least 5 said hydrogenated wax-esters.

13. A formulation according to any one of the preceding claims, characterized in that it comprises 0.5% to 15% by weight of said wax-esters.

14. A cosmetic formulation for use as a skin care cream, a foundation cream, a hair care after-shampoo lotion, or a lip colouring coating, characterized in that it comprises a formulation according to any one of the preceding claims.
